(19) United States
(12) Patent Application Publication
(10) Pub. No.: US 2014/0155275 A1
(43) Pub. Date: Jun. 5, 2014

(54) SEMI-DIGITAL LIGATION ASSAY

(71) Applicants: Bert Vogelstein, Baltimore, MD (US); Kenneth W. Kinzler, Baltimore, MD (US)

(72) Inventors: Bert Vogelstein, Baltimore, MD (US); Kenneth W. Kinzler, Baltimore, MD (US)

(73) Assignee: THE JOHNS HOPKINS UNIVERSITY, Baltimore, MD (US)

(21) Appl. No.: 14/130,776

(22) PCT Filed: Jul. 6, 2012
(86) PCT No.: PCT/US2012/045757

§ 371 Date: Feb. 20, 2014

Related U.S. Application Data
(60) Provisional application No. 61/504,947, filed on Jul. 6, 2011.

(51) Int. Cl. C12Q 1/68

(52) U.S. Cl. CPC C12Q 1/6827; C12Q 1/6883; C12Q 1/6886
USPC 506/2; 435/6.11; 506/9

(57) ABSTRACT

Assays for detecting mutant sequences at particular locations, especially against a background of non-mutant sequences, employ thermocycling ligase reactions. Differentially labeled or sized probes can be used to distinguish wild-type and mutant sequences. Physico-chemical properties of the probes can be critical to successful detection. Mutation detection can be used for diagnosis, monitoring, or prognosticating diseases such as cancers.

The invention was made using funds from the U.S. government. The U.S. government retains certain rights in the invention according to the terms of grants from the National Institutes of Health CA 43460, CA 57345, and CA 62924.

### TECHNICAL FIELD OF THE INVENTION

This invention is related to the area of genetic markers. In particular, it relates to methods for detecting particular nucleic acid sequences. The nucleic acid sequences may be markers, for example markers for cancer or other diseases.

### SUMMARY OF THE INVENTION

According to one aspect of the invention mutations at a selected location in a nucleotide sequence are detected. A reaction mixture is formed of a test sample comprising: 200 or fewer molecules of analyte nucleic acid; a probe complementary to a wild-type sequence at the selected location and adjacent to and proximal to the selected location; a probe complementary to a mutant sequence at the selected location and adjacent to and proximal to the selected location; an anchoring oligonucleotide which is complementary to the analyte nucleic acid adjacent to and distal to the selected location; and a thermotolerant DNA ligase. The probes complementary to the wild-type and mutant sequences are labeled with distinct fluorescent moieties. Or the probes complementary to the wild-type and mutant sequences are of distinct lengths. Or the probes complementary to the wild-type and mutant sequences have distinct fluorescent moieties and distinct lengths. The reaction mixture is thermocycled such that anchoring oligonucleotides are ligated to an appropriate probe reflecting hybridization of the appropriate probe to the analyte nucleic acid. Ligation products are thereby formed. The ligation products are separated on a gel, or the distinct fluorescent moieties are detected, or the distinct fluorescent moieties on the separated ligation products are detected on the gel.

According to another aspect of the invention mutations at a selected location in a nucleotide sequence are detected. An analyte nucleic acid is asymmetrically amplified using a first and second primer to form a test sample. The first primer is in excess of the second primer. A reaction mixture is formed by contacting 200 or fewer molecules of analyte nucleic acid of the test sample; a probe complementary to a wild-type sequence at the selected location and adjacent to and proximal to the selected location; a probe complementary to a mutant sequence at the selected location and adjacent to and proximal to the selected location; an anchoring oligonucleotide which is complementary to the analyte nucleic acid adjacent to and distal to the selected location; and a thermotolerant DNA ligase. The probe that is complementary to the mutant sequence has a Tm of 32 to 36 deg C. The probe that is complementary to the wild-type sequence has a Tm of 32 to 38 deg C. The anchoring oligonucleotide has a Tm of 36 to 44 deg C., as assessed by the oligocalc algorithm. The probe complementary to the mutant sequence comprises one or more locked nucleic acid nucleotides. The wild-type and mutant probes are labeled with distinct fluorescent moieties, or the wild-type and mutant probes are of distinct lengths, or the wild-type and mutant probes have distinct fluorescent moieties and distinct lengths. The reaction mixture is thermocycled such that anchoring oligonucleotides are ligated to an appropriate probe reflecting hybridization of the appropriate probe to the analyte nucleic acid. Ligation products are thereby formed. The ligation products are separated on a gel, or the distinct fluorescent moieties are detected, or the distinct fluorescent moieties are detected on the separated ligation products on the gel.

These and other embodiments Which will be apparent to those of skill in the art upon reading the specification provide the art with methods for assessing, characterizing, and detecting genetic markers, such as cancer markers. In particular, it provides methods for detecting known sequences that may be rare in a test sample.

### BRIEF DESCRIPTION OF THE DRAWINGS

FIG. 1 provides a schematic of a capture strategy. Overlapping oligonucleotides flanked by universal sequences complimentary to the 169 genes listed in FIG. 5 (Table S1) were synthesized on an array. The oligonucleotides were cleaved off the array, amplified by PCR with universal primers, ligated into concatamers and amplified in an isothermal reaction. They were then bound to nitrocellulose filters and used as bait for capturing the desired fragments. An Illumina library was constructed from the sample DNA. The library was denatured and hybridized to the probes immobilized on nitrocellulose. The captured fragments were eluted, PCR amplified and sequenced on an Illumina GAIIX instrument.

FIGS. 2A-2B show a ligation assays used to assess KRAS (v-Ki-ras2 Kirsten rat sarcoma viral oncogene homolog) and GNAS (guanine nucleotide binding protein (G protein), alpha stimulating activity polypeptide 1) mutations. (FIG. 2A) Schematic of the ligation assay. Oligonucleotide probes complementary to either the WI or mutant sequences were incubated with a PCR product containing the sequence of interest. The WT- and mutant-specific probes were labeled with the fluorescent dyes 6-FAM and HEX, respectively, and the WT-specific probe was 11 bases longer than the mutant-specific probe. After ligation to a common anchoring primer, the ligation products were separated on a denaturing polyacrylamide slab gel. Further details of the assay are provided in the Materials and Methods, (FIG. 2 B) Examples of the results obtained with the ligation assay in the indicated patients. Templates were derived from DNA of normal duodenum or IPMN tissue. Each lane represents the results of ligation of one of four independent PCR products, each containing 200 template molecules. The probe in the left panel was specific to the GNAS R201H mutation and the probe on the right panel was specific for the GNAS R201C mutation.

FIG. 3 shows BEAMing assays used to quantify mutant representation. PCR was used to amplify KRAS or GNAS sequences containing the region of interest (KRAS codon 12 and GNAS codon 201). The PCR-products were then used as templates for BEAMing, in which each template was converted to a bead containing thousands of identical copies of the templates (34). After hybridization to Cy3- or Cy5-labeled oligonucleotide probes specific for the indicated WI or mutant sequences, respectively, the beads were analyzed by flow cytometry. Scatter plots are shown for templates derived from the DNA of IPMN 130 or from normal spleen. Beads containing the WT or mutant sequences are widely separated in the scatter plots, and the fraction of mutant-containing beads are indicated. Beads whose fluorescence spectra lie between the WT and mutant-containing beads result from inclusion of both WT and mutant templates in the aqueous nanocompartments of the emulsion PCR.

FIGS. 4A-4C show IPMN morphologies. (FIG. 4A) H&E-stained section of a formalin-fixed, paraffin embedded sample (shows two apparently independent IPMNs with distinct morphologies located close to one another. The IPMN of gastric epithelial subtype (black arrow) harbored a GNAS R201C and a KRAS G12′V while the IPMN showing the intestinal subtype (red arrow) contained a GNAS R201C mutation but no KRAS mutation. (FIG. 4B) H&E stained section of a different, typical IPMN (FIG. 4C) Same IPMN as in FIG. 4B after microdissection of the cyst wall.

FIG. 5. (Table S1.) Genes analyzed by massively parallel sequencing in IPMN cyst fluids.

FIG. 6. (Table S2.) Characteristics of patients with IPMNs analyzed in this study, including GNAS and KRAS mutation status.

FIG. 7. (Table S3) Characteristics of patients with cyst types other than IPMN, including GNAS and KRAS mutation status.

FIG. 8. (Table S4.) Quantification of mutations in selected IPMNs containing both GNAS and KRAS mutations.

FIG. 9. (Table S5.) Comparison of mutational status in DNA from IPMNs and pancreatic adenocarcinomas from the same patients.

FIG. 10. (Table S6.) Oligonucleotide primer and probe sequences (SEQ ID NO: 4-38).

### DETAILED DESCRIPTION OF THE INVENTION

The inventors have found a sensitive way of assaying for mutant nucleic acid sequences that may be infrequent in a population of such sequences. The assay is particularly useful in situations where mutations occur at a small number of locations. Under such circumstances, probes can be made for mutations that are known to occur. Probes can also be made for the wild-type nucleic acid sequence, which may be the dominant sequence in a population of sequences.

In order to find rare sequences in a population of similar but different sequences, one can separate a test sample into multiple aliquots with a ceiling on the number of analyte nucleic acid molecules per aliquot. The ceiling may be 1000, 750, 500 250, 200, 150, 100, 100, or 50 molecules, for example. Even if a nucleic acid analyte is present in a test sample in an amount too low for detection by an assay, by dividing the test sample into aliquots, a higher ratio of desired analyte to background analytes can be achieved. In order to increase the reliability and sensitivity of detecting rare sequences, the original population of analyte molecules can be amplified, for example using polymerase chain reaction or rolling circle amplification. Asymmetric amplification of an analyte nucleic acid may be used. A first and second primer can be used, and the first primer is in excess of the second primer.

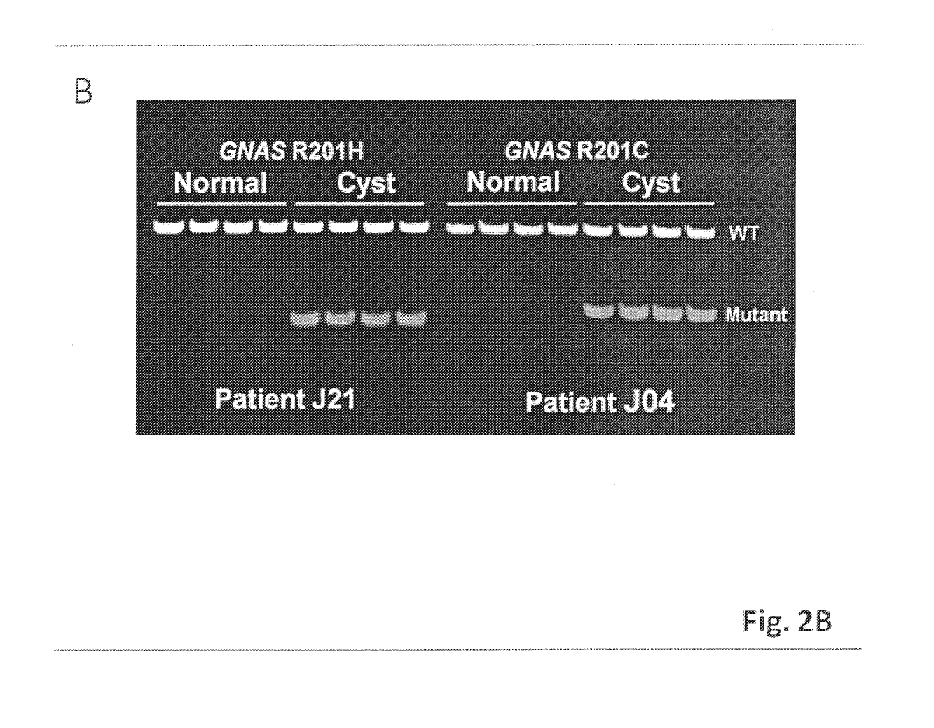

Each assay sample can be contacted with three oligonucleotides. The first oligonucleotide is a probe complementary to a wild-type sequence at a selected location and adjacent to and proximal to the selected location. The second oligonucleotide is a probe complementary to a mutant sequence at the selected location and adjacent to and proximal to the selected location. The third oligonucleotide is an anchoring oligonucleotide Which is complementary to the analyte nucleic acid adjacent to and distal to the selected location. A schematic graphically representing these three oligonucleotides is provided in FIG. 2A.

The probes complementary to the wild-type and mutant sequences can optionally be labeled with distinct fluorescent moieties. The probes complementary to the wild-type and mutant sequences can optionally be of distinct lengths. Alternatively, the probes complementary to the wild-type and mutant sequences can optionally have both distinct fluorescent moieties and distinct lengths. These differences allow the rare reaction product to be more easily detected among a background of predominant reaction products. For example, if a sample is heterozygous for a mutation at a particular locus, these differences in probes facilitate the detection of the two products.

The probes may have particular physical-chemical characteristics, making them better at binding in a discriminating fashion to the template molecules. The probe complementary to the mutant sequence may have a Tm of 32 to 36 deg C. The probe complementary to the wild-type sequence may have a Tm of 32 to 38 deg C. The anchoring oligonucleotide may have a Tm of 36 to 44 deg C., as assessed by the oligocalc algorithm (available from Northwestern University, Chicago, Ill., Biotools)).

Other enhancements to the physical chemistry of the probes may be used. For example, the probe complementary to the mutant sequence may comprise one or more locked nucleic acid nucleotides. The probe may comprise three locked nucleic acid nucleotides, The locked nucleotide residues may be at positions -2,-3, and -7, wherein position 0 is the selected location where a mutation may be present.

The assay employs a thermotolerant DNA ligase, which is stable at various temperatures through which the reaction is cycled. While one particular cycling schedule is described below, others can be used, which may vary the precise times and or temperatures. The cycling to high temperatures, permits the melting off of a ligated single strand product from the template molecule, permitting another set of probes and anchoring oligonucleotides to anneal and be ligated together after the assay is cooled to a suitable temperature for annealing. By cycling, one analyte molecule can serve as a template for a number of ligated oligonucleotide products. Probes that hybridize adjacent to the oligonucleotide on an analyte template molecule can be ligated to each other by the thermotolerant DNA ligase.

Ligation products can be separated on a gel or other medium or using another technique that separates on the basis of size and/or charge. These may use chromatography, spectroscopy, flow cytometry, or other suitable technique. The distinct fluorescent moieties can be detected using any technique for imaging or observing fluorescence. The two types of techniques, for detecting size and fluorescence, can be used simultaneously or sequentially.

Probes and/or primers may contain the wild-type or a mutant sequence. These can be used in a variety of different assays, as will be convenient for the particular situation, Selection of assays may be based on cost, facilities, equipment, electricity availability, speed, reproducibility, compatibility with other assays, invasiveness of sample collection, sample preparation, etc.

Any of the assay results may be recorded or communicated, as a positive act or step. Communication of an assay result, diagnosis, identification, or prognosis, may be, for example, orally between two people, in writing, whether on paper or digital media, by audio recording, into a medical chart or record, to a second health professional, or to a patient. The results and/or conclusions and/or recommendations based on the results may be in a natural language or in a machine or other code. Typically such records are kept in a confidential manner to protect the private information of the patient.

Collections of any of probes, primers, control samples, thermotolerant ligase, and reagents can be assembled into a kit for use in the methods. The reagents can be packaged with instructions, or directions to an address or phone number from which to obtain instructions. An electronic storage medium may be included in the kit, whether for instructional purposes or for recordation of results, or as means for controlling assays and data collection.

Control samples can be obtained from a tissue that is not apparently diseased, for example from the patient. Alternatively, control samples can be obtained from a healthy individual or a population of apparently healthy individuals. Control samples may be from the same type of tissue or from a different type of tissue than the test sample.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

### Patients and Specimens

The present study was approved by the Institutional Review Boards of Johns Hopkins Medical Institutions, Memorial Sloan Kettering Cancer Center and the University of Indiana. We included individuals in Whom pancreatic cyst fluid samples from pancreatectomy specimens and/or fresh frozen tumor tissues were available for molecular analysis. Relevant demographic, clinicopathologic data were obtained from prospectively maintained clinical databases and correlated with mutational status.

Pancreatic cyst fluids were harvested in the Surgical Pathology suite from surgically resected pancreatectomy specimens with a sterile syringe. Aspirated fluids were stored at −80° C. within 30 min of resection. Fresh-frozen tissue specimens of surgically resected cystic neoplasms of the pancreas were obtained through a prospectively maintained Johns Hopkins Surgical Pathology Tumor Bank. These lesions as well as normal tissues were macrodissected using serial frozen sections to guide the trimming of OCT embedded tissue Hocks to obtain a minimum neoplastic cellularity of 80%. Formalin-fixed and paraffin-embedded archival tissues from surgically resected pancreata were sectioned at 6 μm, stained with hematoxylin and eosin, and dissected with a sterile needle on a SMZ1500 stereomicroscope Nikon). An estimated 5,000-10,000 cells were microdissected from each lesion. Lesions were classified as IPMNs, MCNs, or SCAs using standard criteria (53) IPMNs were subtyped by internationally accepted criteria (54).

### DNA Purification

DNA was purified from frozen cyst walls using an AllPrep kit (Qiagen) and from forrmalin-fixed, paraffin-embedded sections using the QIAamp DNA FFPE tissue kit (Qiagen) according to the manufacturer's instructions. DNA was purified from 250 μL of cyst fluid by adding 3 ml RLTM buffer (Qiagen) and then binding to an AllPrep DNA column (Qiagen) following the manufacturer's protocol. DNA was quantified in all cases with qPCR, employing primers and conditions as described (55).

### Illumina Library Preparation

Cyst fluid DNA was first quantified through real-time PCR using primers specific for repeated sequences in DNA (LINE) as described (56). A minimum of 100 ng DNA from cyst fluid was used to make IIlumina libraries according to manufacturer's protocol with the exception that the amount of adapters was decreased in proportional fashion when a lower amount of template DNA was used. The number of PCR cycles used to amplify the library after ligation of adapters was varied to ensure a yield of ˜5 μg of the final library product for capture.

### Target DNA Enrichment

The targeted region included all of the 3386 exons of 169 cancer related genes and was enriched with custom-made oligonucleotide probes. The design of each oligonucleotide was as follows: 5′-TCCCGCGACGAC—36 bases from the genomic region of interest—GCTGGAGTCGCG-3′ (SEQ ID NO: 1). Probes were designed to capture both the plus and the minus strand of the DNA and had a 33-base overlap. The probes were custom-synthesized on a chip. The oligonucleotides were cleaved from the chip by treatment for five hours with 3 ml 35% ammonium hydroxide at room temperate. The solution was transferred to two 2-ml tubes, dried under vacuum, and re-dissolved in 400 ul RNase and DNase free water. Five ul of the solution were used for PCR amplification with primers complementary to the 12 base sequence common to all probes: 5-TGATCCCGCGACGA*C-3′ (SEQ ID NO: 2), 5′-GACCGCGACTCCAG*C-3′ (SEQ ID NO: 3), with * indicating a phosphorothioate bond. The PCR mix contained 27 ul H₂O, 5 ul template DNA, 2 ul forward primer (25 uM), 2 ul reverse primer (25 uM), 4 ul MgCl₂ (50 ml), 5 ul 10× Platinum Taq buffer (Life Technologies), 4 ul dNTPs (10 mM each) and 1 ul Platinum Taq (SU/ul, Life Technologies). The cycling conditions were: one cycle of 98° C. for 30 s; 35 cycles of 98° C. for 30 s, 40° C. for 30 s, 60° C. for 15 s, 72° C. for 45 s; one cycle of 72° C. for 5 min. The PCR product was purified using a MinElute Purification Column (Qiagen) and end-repaired using End-IT DNA End-Repair Kit (Epicentre) as follows: 34 ul DNA, 5 ul 10× End-Repair Buffer, 5 ul dNTP Mix, 5 ul ATP, 1 ul. End-Repair Enzyme Mix. The mix was incubated at room temperature for 45 minutes, and then purified using a MinElute Purification Column (Qiagen). The PCR products were ligated to form concatamers using the following protocol: 35 ul End-Repaired DNA product, 40 ul 2x 14 DNA ligase buffer, 5 ul T4 DNA ligase (3000 units; Enzymatics Inc.) The mix was incubated at room temperature for 4 hours, then purified using QiaQuick Purification Column (Qiagen), and quantified by absorption at 260 nm.

Replicates of 50 ng of concatenated PCR product were amplified in 25 ul solution using the REPLI-g midi whole genome amplification kit (Qiagen) according to the manufacturer's protocol. The RepliG-amplified DNA (20 ug) was then bound to a nitrocellulose membrane and used to capture DNA libraries as described (57). In general. 5 ug of library DNA were used per capture. After washing, the captured libraries were ethanol precipitated and redissolved in 20 ul TE buffer. The DNA was then amplified in a PCR mix containing 51 ul H₂O, 20 ul 5× Phusion buffer, 5 ul DMSO, 2 ul 10 mM dNTPs, 50 pmol Illumina forward and reverse primers, and 1 ul Hotstart Phusion enzyme (New England Biol_abs) using the following cycling program: 98° C. for 30 sec; 15 cycles of 98° C. for 25 sec., 65° C. for 30 sec, 72° C. for 30 sec; and 72° C. for 5 min. The amplified PCR product was purified using a NucleoSpin column (Macherey Nagel, inc.) according to the manufacturer's suggested protocol except that the NT buffer was not diluted and the DNA bound to the column was eluted in 35 ul elution buffer. The captured library was quantified with realtime PCR with the primers used for grafting to the Illumina sequencing chip.

### Ligation Assay

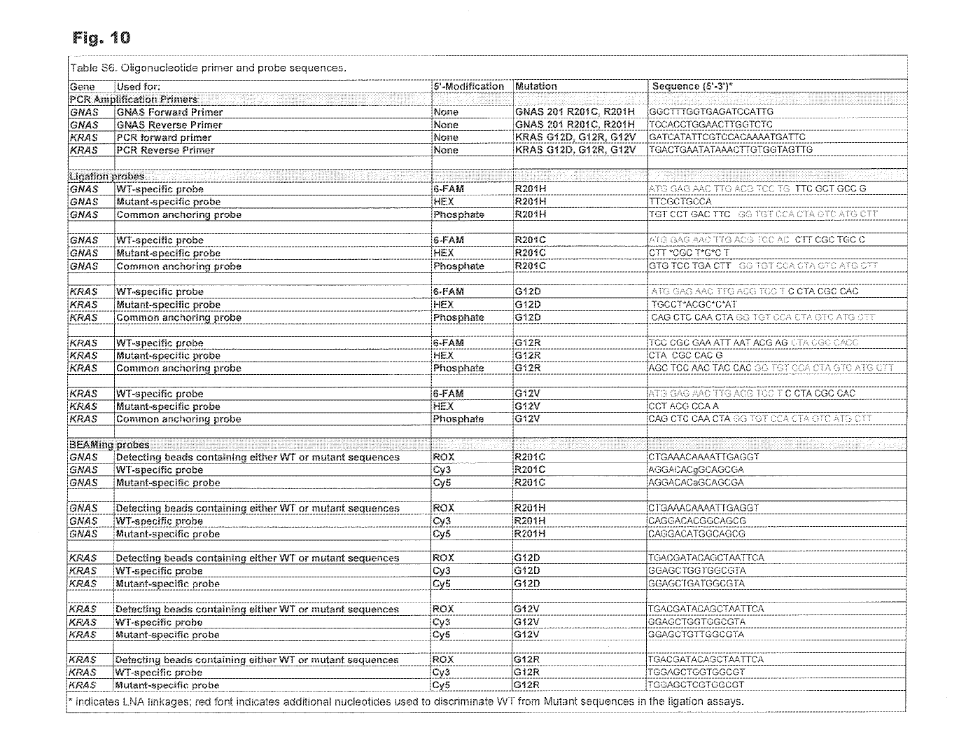

PCR products containing codon 12 of KRAS and codon 201 of GNAS were amplified using the primers described in FIG. 10 (Table S6). Each PCR contained 200 template molecules in 5 ul of 2× Phusion Flash PCR Master Mix (New England Biolabs) and final concentrations of 0.25 uM forward and 1.5 uM reverse primers. Note that the mutant-specific probes sometimes included locked nucleic acid residues (FIG. 10 (Table S6); Exiqon). The following cycling conditions were used: 98° C. for 2 min; 3 cycles of 98° C. for 10 sec., 69° C. for 15 sec, 72° C. for 15 sec; 3 cycles of 98° C. for 10 sec., 66° C. for 15 sec, 72° C. for 15 sec; 3 cycles of 98° C. for 10 sec., 63° C. for 15 sec, 72° C. for 15 sec; 41 cycles of 98° C. for 10 sec., 60° C. for 60 sec. Reactions were performed in at least quadruplicate and each was evaluated independently. Five ul of a solution containing 0.5 ul of Proteinase K. (18.8 mg/ml, Roche,) and 4.5 ul of dH₂O was added to each well and incubated at 60° C. for 30 minutes to inactivate the Phusion polymerase and then for 10 min at 98° C. to inactivate the Proteinase K.

The ligation assay was based on techniques described previously, using thermotolerant DNA ligases (58-61). Each 10 -ul reaction contained 2-ul of PCR product (unpurified), 1 ul of 10× Ampligase buffer (Epicentre), 0.5 ul of Ampligase (512/ul , Epicentre), anchoring primer (final concentration 2 uM), WT-specific primer (final concentration 0.1 uM), and mutant-specific primer (final concentration 0.025 uM). The sequences of these primers are listed in FIG. 10 (Table S6). The following cycling conditions were used: 95° C. for 3 min; 35 cycles of 95° C. for 10 sec., 37° C. for 30 sec, 45° C. for 60 sec. Five ul of each reaction was added to 5 ul of formamide and the ligation products separated on a 10% Urea-Tris-Borate-EDTA gel (Invitrogen) and imaged with an Amersham-GE Typhoon instrument (GE Healthcare)

### BEAMing Assays

These were performed as described (62) using the PCR products generated for the ligation assay as templates and the oligonucleotides listed in FIG. 10 (Table S6) as hybridization probes.

### Statistical Analysis

Fisher's exact tests were used to compare the differences between proportions and Wilcoxon Rank Sum tests were used to compare differences in mutational status by age. Confidence intervals for the prevalence of mutations were estimated using the binomial distribution. To compare the prevalence of mutations in grossly distinct IPMNs to adjacent locules within a single grossly distinct IPMN, we compared the probability of observing given KRAS or GNAS mutation in the 111 distinct IPMNs to conditional probability that given the first locule sequenced contained a specific KRAS or GNAS mutation all other locules contained the same KRAS or GNAS mutations. The probabilities of GNAS or KRAS mutations occurring by chance was calculated using a binomial distribution and the previously estimated mutation rates of tumors or normal cells (30). STATA version 11 vas used for all statistical analysis (63).

### EXAMPLE 2

### Massively Parallel Sequencing of 169 Genes in Cyst Fluid DNA

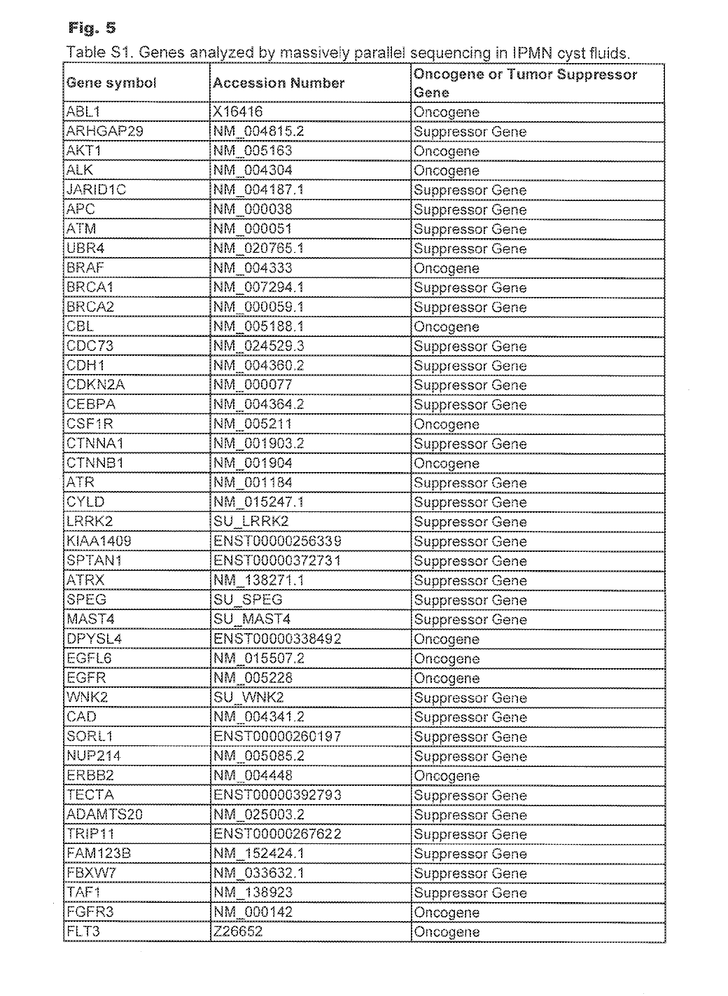

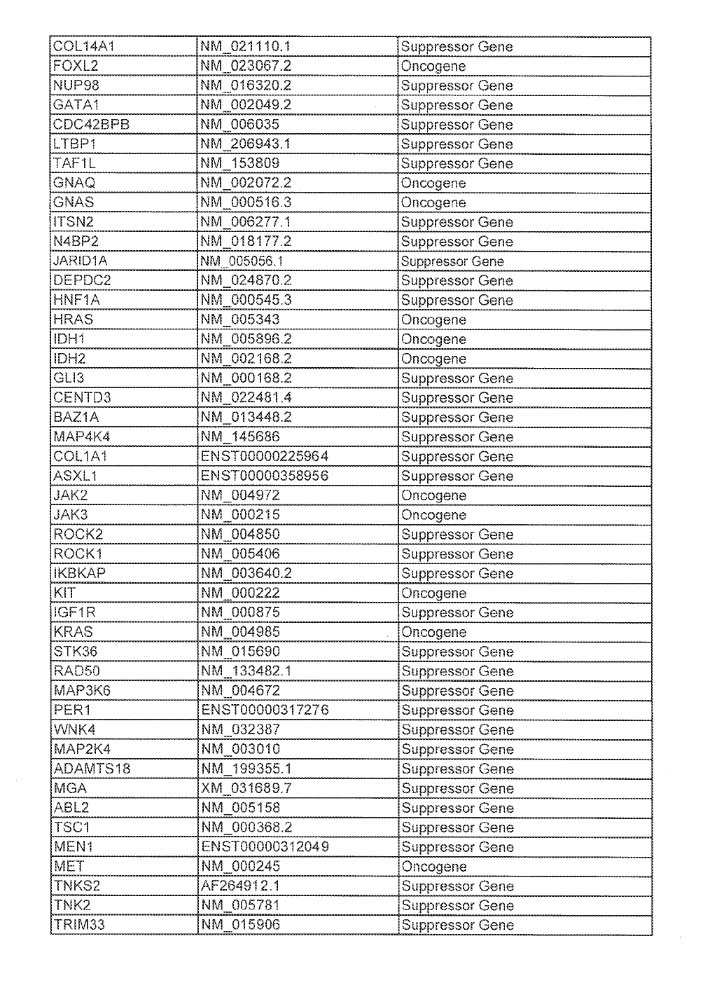

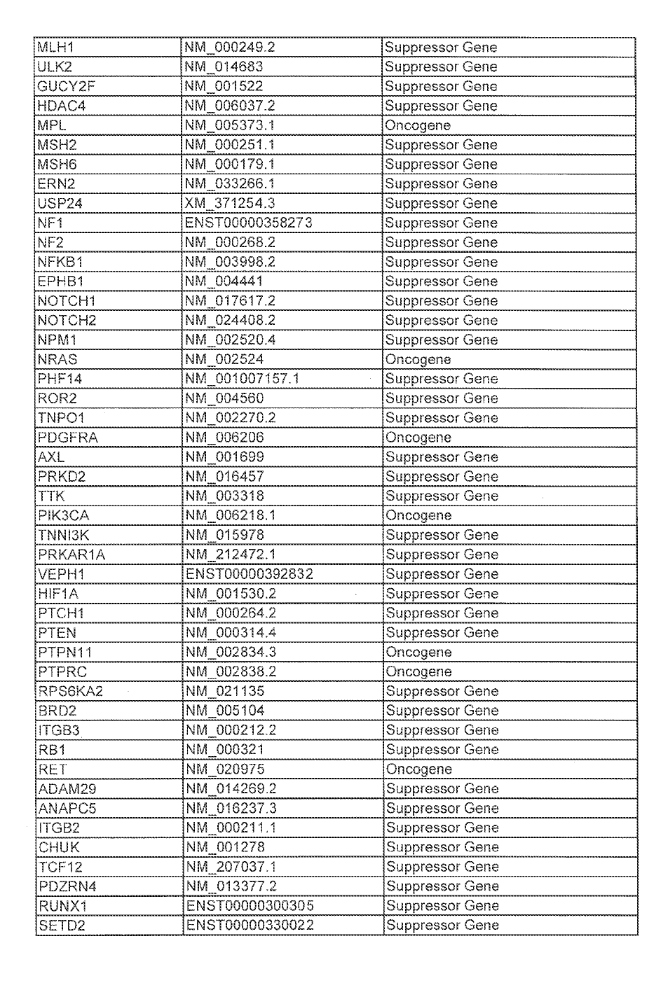

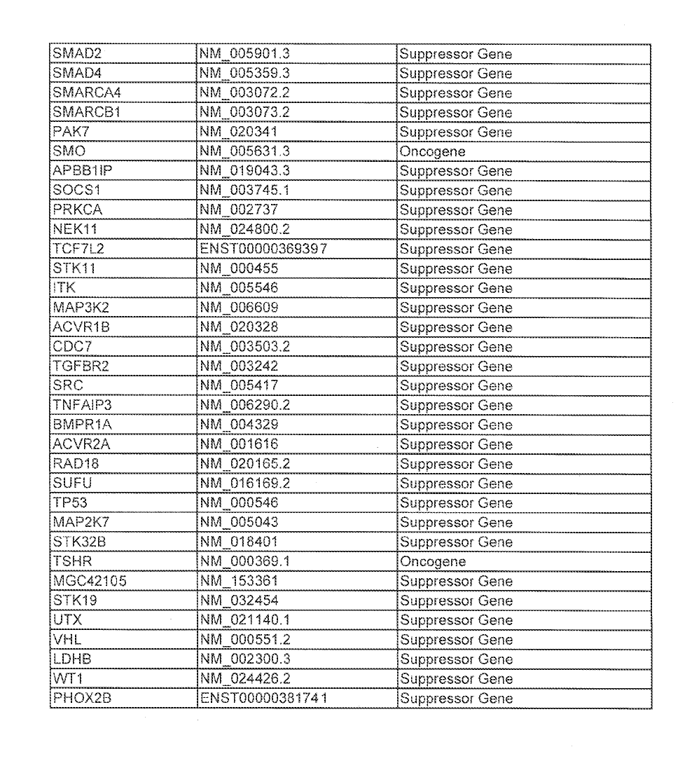

To initiate this study, we determined the sequences of 169 presumptive cancer genes in the cyst fluids of 19 IPMNs, each obtained from a different patient. Thirty-three of the 169 were oncogenes and the remainder were tumor suppressor genes. Though only a tiny subset of these 169 genes were known to be mutated in PDAs, all were known to be frequently mutated in at least one solid tumor type (FIG. 5 (Table S1). We additionally sequenced these genes in normal pancreatic, splenic or intestinal tissues of the same patients to determine which of the alterations identified were somatic. We chose to use massively parallel sequencing rather than Sanger sequencing for this analysis because we did not know what fraction of DNA purified from the cyst fluid was derived from neoplastic cells. Massively parallel sequencing has the capacity to identify mutations present in 2% or more of the studied cells while Sanger sequencing often requires >25% neoplastic cells for this purpose. IPMNs are by definition connected with the pancreatic duct system and the cyst fluid containing cellular debris and shed DNA from the neoplastic cells can be expected to be admixed with that of the cells and secretions derived from normal ductal epithelial cells.

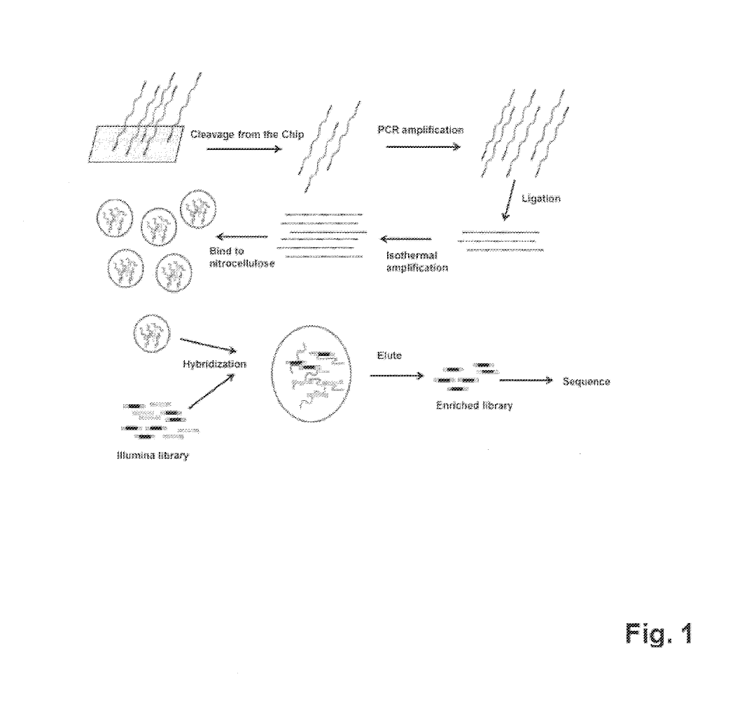

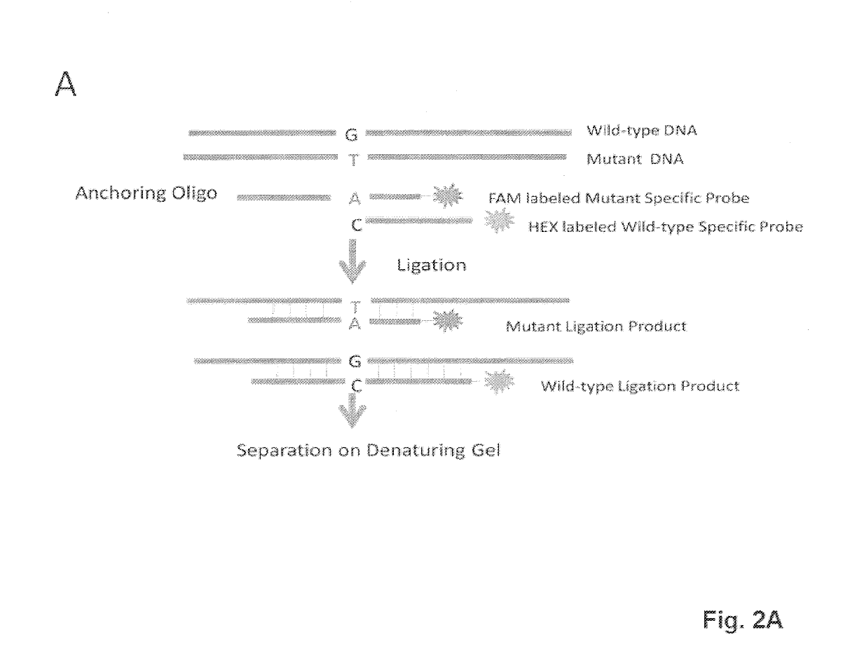

We devised a strategy to capture sequences of the 169 genes from cyst fluid DNA (FIG. 1). In brief, 244,000 oligonucleotides, each 60 bp in length and in aggregate covering the exonic sequences of all 169 genes, were synthesized in parallel using phosphoramadite chemistry on a single chip synthesized by Agilent Technologies. After removal from the chip, the oligonucleotide sequences were amplified by PCR and ligated together. Multiple displacement amplification was then used to further amplify the oligonucleotides, which were then bound to a filter. Finally, the filter was used to capture complementary DNA sequences from the cyst fluids and corresponding normal samples, and the captured DNA was subjected to massively parallel sequencing.

The target region corresponding to the coding exons of the 169 genes encompassed 584,871 bp. These bases were redundantly sequenced, with 902±411 (mean±1 SD) fold-coverage in the 38 samples sequenced (19 IPMN cyst fluids plus 19 matched DNA samples from normal tissues of the same patients). This coverage allowed us to confidently detect somatic mutations present in >5% of the template molecules.

There were only two genes mutated in more than one IPMN-KRAS, which was mutated in 14 of the 19 IPMNs, and GNAS, which was mutated in 6 IPMNs. The mutations in GNAS all occurred at codon 201, resulting in either a R201H or R201C substitution. GNAS is a well-known oncogene that is mutated in pituitary and other uncommon tumor types (16-19). However, such mutations have rarely been reported in common epithelial tumors (20-22). In pituitary tumors, mutations cluster at two positions—codons 201 and 227 (16, 19). This clustering provides extraordinary opportunities for diagnosis, similar to that of KRAS. For example, the clustering of KRAS mutations has facilitated the design of assays to detect mutations in tumors of colorectal cancer patients eligible for therapy with antibodies to EGFR (23). All twelve KRAS mutations identified through massively parallel sequencing of cyst fluids were at codon 12, resulting in a G12D, G12V, or G12R amino acid change. KRAS mutations at codon 12 have previously been identified in the vast majority of PDAs as well as in 40 to 60% of IPMNs (24-29). GNAS mutations have not previously been identified in pancreatic cysts or in PDAs.

### EXAMPLE 3

### Frequency of KRAS and GNASA Mutations in Pancreatic Cyst Fluid DNA

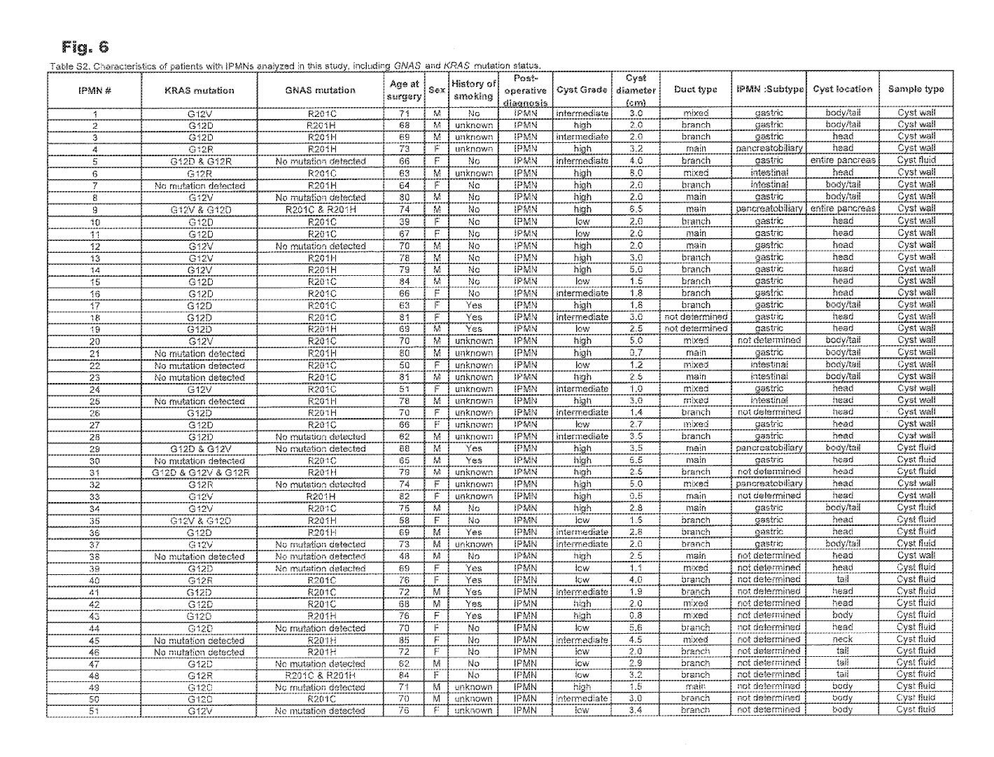

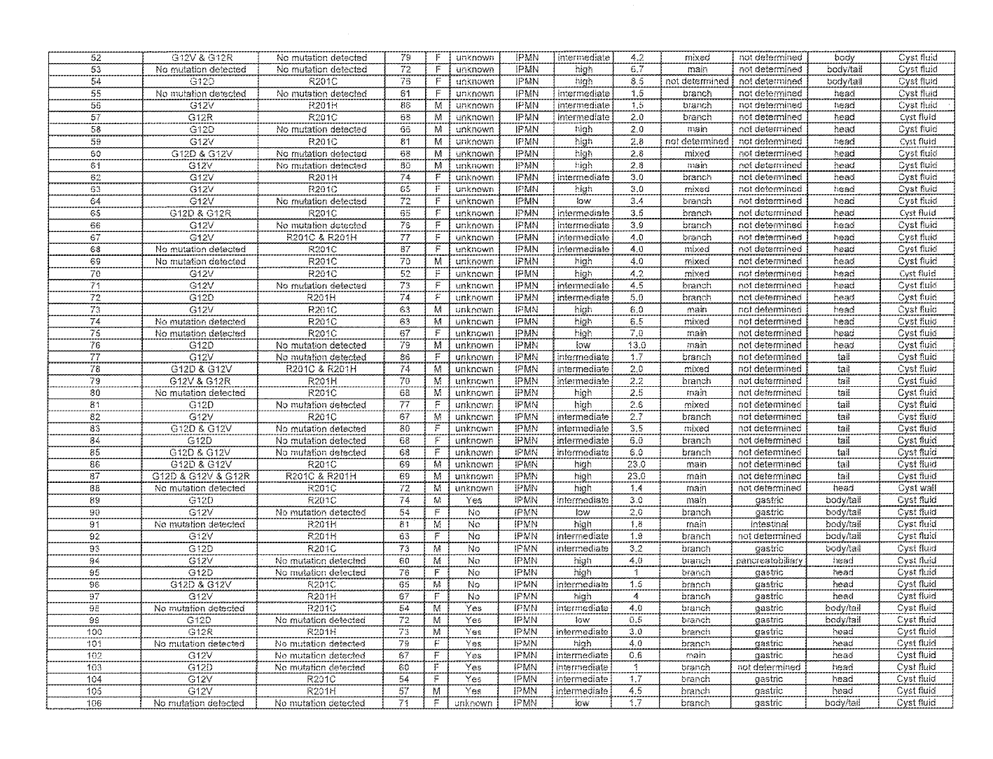

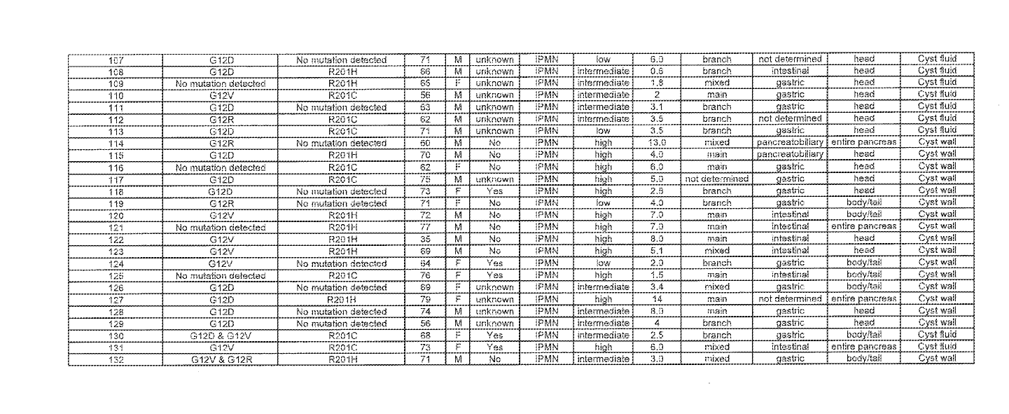

We next determined the frequency of KRAS codon 12 and GNAS codon 201 mutations in a larger set of IPMNs. The clinical characteristics of all IPMNs analyzed in this study are listed in FIG. 6 (Table S2). To ensure that the analyses were performed robustly, we carried out preliminary experiments with cyst fluids from patients with known mutations based on the massively parallel sequencing experiments described above. We tested several methods for purifying DNA from often viscous cyst fluids and used the optimum method for subsequent experiments. Quantitative PCR was used to determine the number of amplifiable template molecules recovered with this procedure. In eight cases, we compared pelleted cells to supernatants derived from the same cyst fluid samples and found that the fraction of mutant templates in both compartments was similar. On the basis of these results, we purified DNA from 0.2.5 ml of whole cyst fluid (cells plus supernatant) and, as assessed by quantitative PCR, recovered an average of 670±790 ng of usable DNA.

For each of 84 cyst fluid samples (an independent cohort of 65 patients plus the 19 patients whose fluids had been studied by massively parallel sequencing), we analyzed ˜800 template molecules for five distinct mutations, three at KRAS codon 12 and two within GNAS codon 201 (see Materials and Methods). A PCR/ligation method that had the capacity to detect one mutant template molecule among 200 normal (wild-type, WT) templates was used for these analyses (FIG. 2A). We identified GNAS' and KRAS mutations in 61% and 82% of the IPMN fluids, respectively (representative examples in FIG. 2B). In those samples without GALAS codon 201 mutations, we searched for GNAS codon 227 mutations, but did not find any. We also analyzed macro- and microdissected frozen or paraffin-embedded cyst walls from an independent collection of 48 surgically resected IPMNs, and similarly identified a high prevalence of GNAS (75%) and KRAS (79%) mutations. In aggregate, 66% of 132 IPMNs harbored a GNAS mutation, 81% harbored a KRAS mutation, slightly more than half (51%) harbored both GNAS and KRAS mutations, while at least one of the two genes was mutated in 96.2% (FIG. 6 (Table S2)). Given background mutation rates in tumors or normal tissues (30), the probability that either GNAS or KRAS mutations occurred by chance alone was less than 10⁻³⁰. There were no significant correlations between the prevalence of KRAS or GAS mutations and age, sex, or smoking history of the patients (P>0.05) (Table 1). Small (<3 cm) as well as larger cysts had similar fractions of both KRAS and GNAS mutations and the location of the IPMN (head, body, or tail) did not correlate with the presence of mutation in either gene (Table 1). GNAS and KRAS mutations were present in low-grade as well as in high-grade IPMNs. The prevalence of KRAS mutations was higher in lower grade lesions (P=0.03) whereas the prevalence of GNAS mutations was somewhat higher in more advanced lesions (P=0.11) (Table 1). GNAS, as well as KRAS mutations were present in each of the three major histologic types of IPMNs—intestinal, pancreatobiliary, and gastric. However, the prevalence of the mutations varied across the histological types (P<0.01 for both KRAS and GNAS). GNAS mutations were most prevalent in the intestinal subtype (100%), KRAS mutations had the highest frequency (100%) in the pancreatobiliary subtype and had the lowest frequency (42%) in the intestinal subtype (Table 1).

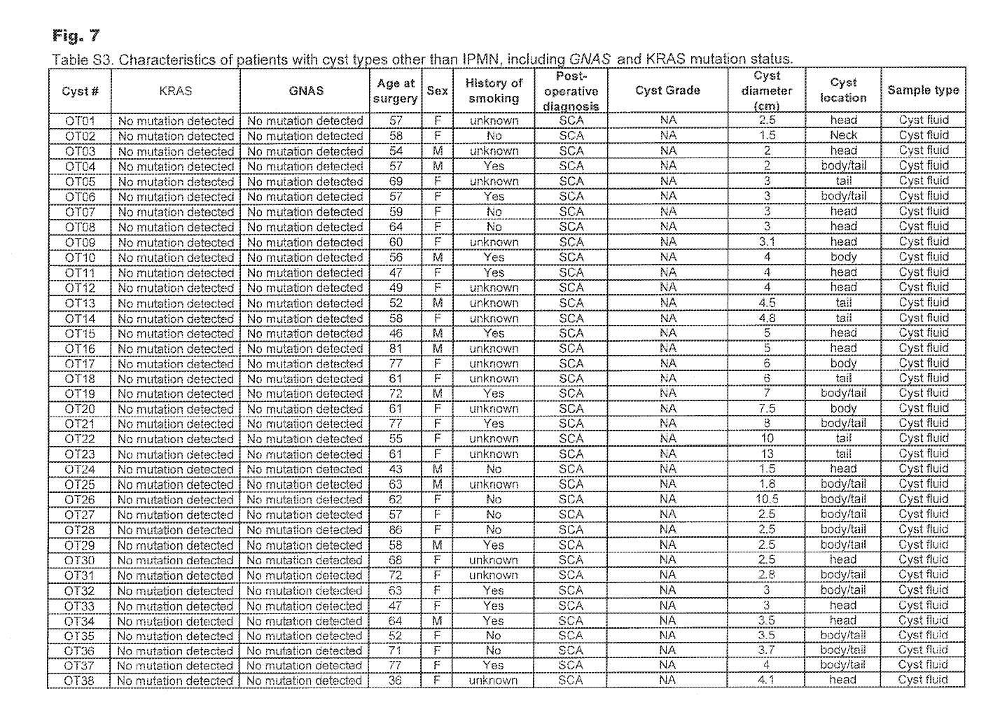

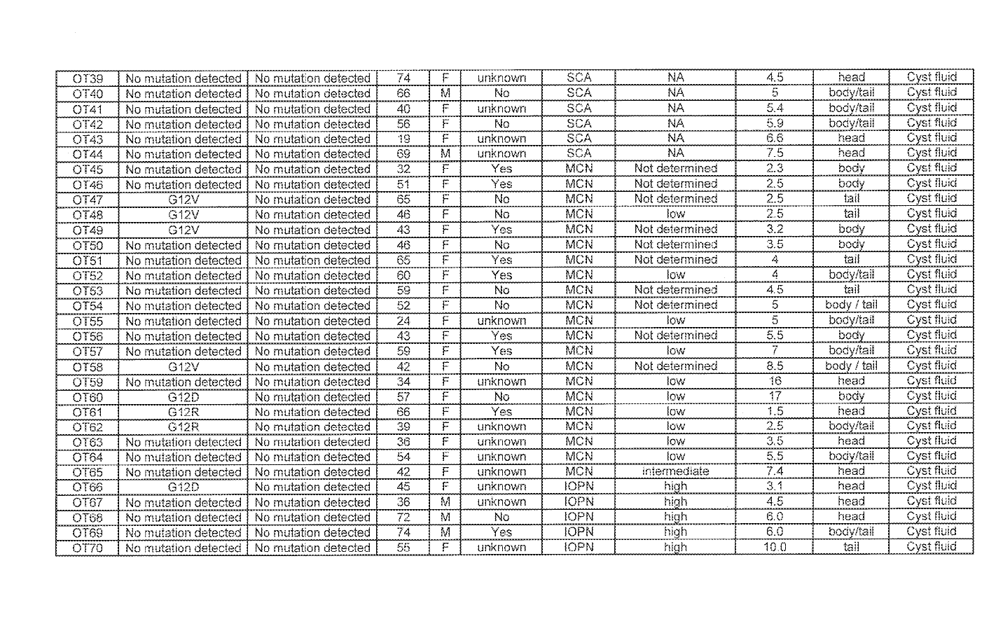

We then determined whether GNAS mutations were present in SCAs, a common but benign type of pancreatic cystic neoplasm. We examined a total of 44 surgically resected SCAs, each from a different patient (42 cyst fluids and 2 cyst walls). Many of these cysts were surgically resected because they clinically mimicked an IPMN. They would have likely not been surgically excised had they been known to be SCAs. The SCAs averaged 5.0±2.8 cm in maximum diameter (FIG. 7 (Table S3))similar to the IPMNs (4.4±3.7 maximum diameter, FIG. 6 (Table S2)). There was little difference in the locations of the SCAs and IPMNs within the pancreas (FIGS. 6 and 7 (Tables S2 and S3)). However, no GNAS or KRAS mutations were identified in the SCAs, in marked contrast to the IPMNs (p<0.001, Fisher's Exact Test). GNAS mutations were also not identified in any of 21 MCNs (p=0.005 when compared to IPMNs, Fisher's Exact Test), although KRAS mutations were found in 33% of MCNs (FIG. 7 (Table S3)). GNAS mutations were also not identified in five examples of an uncommon type of cyst, called intraductal oncocytic papillary neoplasm (IOPN), with characteristic oncocytic features (FIG. 7 (Table S3)).

TABLE 1
Correlations between mutations and clinical and histopathologic parameters of IPMNs

| | | N, | KRAS mutation | | P- | GNAS mutation | | |
|---|---|---|---|---|---|---|---|---|
| | | total | N | % | value | N | % | P-value |
| Age in years | <65 years | 29 | 22 | 75.9 | 0.42 | 18 | 62.1 | 0.62 |
| | ≧65 years | 103 | 85 | 82.5 | | 69 | 67 | |
| Gender | Male | 70 | 58 | 82.9 | 0.58 | 51 | 72.9 | 0.07 |
| | Female | 62 | 49 | 79 | | 36 | 58.1 | |
| History of | Yes | 25 | 21 | 84 | 0.77 | 17 | 68 | 0.85 |
| smoking | No | 37 | 30 | 81.1 | | 26 | 70.3 | |
| Grade | Low | 23 | 20 | 87 | 0.43 | 11 | 47.8 | 0.04 |
| | Intermediate | 51 | 46 | 90.2 | (low vs. | 34 | 66.7 | (low vs. |
| | High | 58 | 41 | 70.7 | others) | 42 | 72.4 | others) |
| Duct type | Main | 35 | 23 | 65.7 | 0.002 | 24 | 68.6 | 0.37 |
| | Branch | 64 | 58 | 90.6 | (main | 38 | 59.4 | (main |
| | Mixed | 28 | 21 | 75 | vs. branch) | 20 | 71.4 | vs. branch) |
| Subtype | gastric | 52 | 45 | 86.5 | 0.02 | 34 | 65.4 | 0.002 |
| | Pancreatobiliary | 7 | 7 | 100 | (panc. vs | 3 | 42.9 | (panc. vs |
| | Intestinal | 13 | 6 | 46.2 | intestinal) | 13 | 100 | intestinal) |
| Diameter | <3 cm | 62 | 49 | 79 | 0.58 | 41 | 66.1 | 0.96 |
| | ≧3 cm | 70 | 58 | 82.9 | | 46 | 65.7 | |
| Location | Proximal (head) | 77 | 64 | 83.1 | 0.44 | 53 | 68.8 | 0.38 |
| | Distal (body, tail) | 49 | 38 | 77.6 | (prox. vs | 30 | 61.2 | (prox. vs) |
| | Proximal and distal | 6 | 5 | 83.3 | distal) | 4 | 66.7 | distal) |
| Associated | Yes | 24 | 18 | 75 | 0.4 | 18 | 75 | 0.3 |
| cancer | No | 108 | 89 | 82.4 | | 69 | 63.9 | |

### EXAMPLE 4

### IPMN Polyclonality

KRAS G12D, G12V, and G12R mutations were found in 43%, 39%, and 13% of IPMNs, respectively (FIG. 6 (Table S2)). A small fraction (11%) of the IPMNs contained two different KRAS mutations and 2% contained three different mutations. Likewise, GNAS R201C, and GNAS R201H mutations were present in 39% and 32% of the IPMNs, respectively, and 4% of IPMNs had both mutations (FIG. 6 (Table S2)). More than one mutation in KRAS in IPMNs has been observed in prior studies of IPMNs (31-33) and the multiple KRAS and GNAS mutations are suggestive of a polyclonal origin of the tumor.

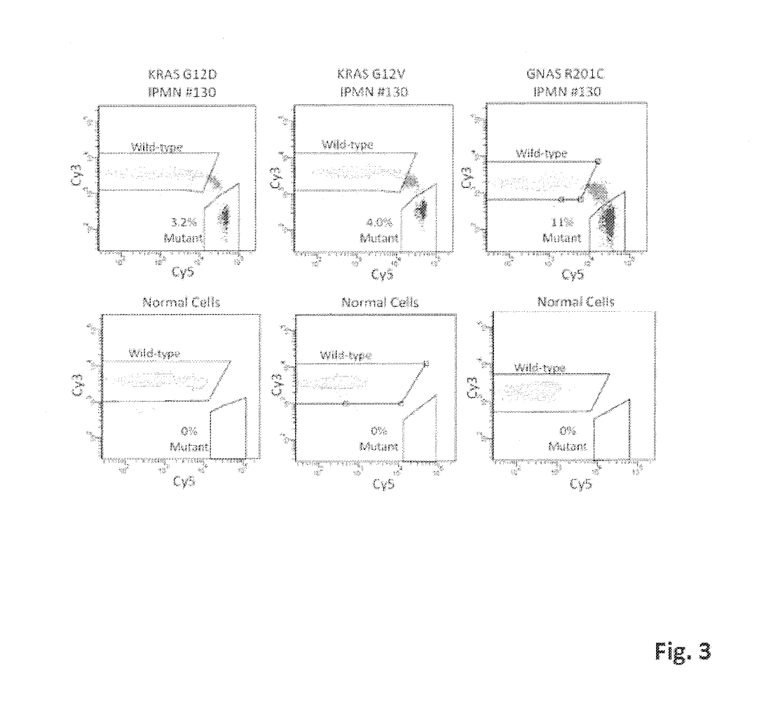

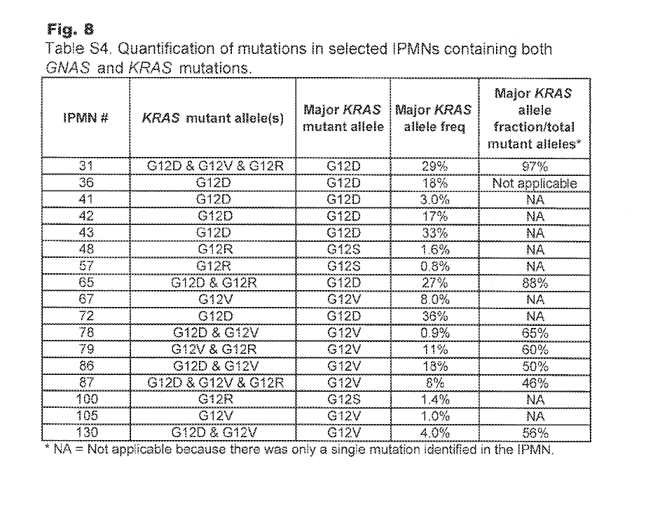

We investigated clonality in more detail by precisely quantifying the levels of mutations in the subset of cyst fluids containing more than one mutation of the same gene. To accomplish this, we used a technique called BEAMing (34) Through this method, individual template molecules are converted into individual magnetic beads attached to thousands of molecules with the identical sequence. The beads are then hybridized with mutation-specific probes and analyzed by flow cytometry (FIG. 3). The analysis of 17 IPMN cyst fluids, each with mutations in both KRAS and GNAS, showed that the fraction of mutant alleles varied widely, ranging from 0.8% to 45% of the templates analyzed. There was an average of 12.8%±12.2% mutant alleles of KRAS and an average of 24.4±13.1% mutant alleles of GNAS in the 17 IPMN cyst fluids examined (FIG. 8 (Table S4)). In two of the seven IPMNs with more than one KRAS mutation, there was a predominant mutant that out-numbered the second KRAS mutant by >5:1 (FIG. 8 (Table S4)). Similarly, two of the four cases harboring two different GNAS mutations had a predominant mutant (FIG. 8 (Table S4)). In the other cases, the different mutations in KRAS (or GNAS) were distributed more evenly (FIG. 8 (Table S4)). These data support the idea that cells within a subset of IPMNs had undergone independent clonal expansions, giving rise to apparent polyclonality (35).

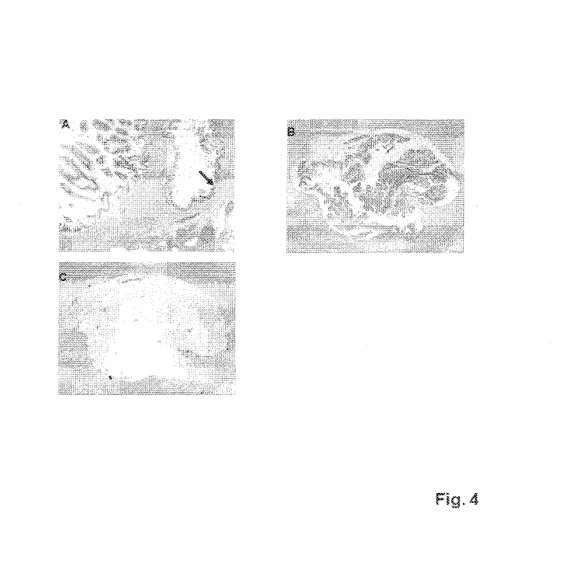

IPMNs are often multilocular or multifocal in nature, looking much like a bunch of grapes (FIG. 4A) (36). To determine the relationship between cyst locules individual grapes) and cyst fluid, we microdissected the walls from individual locules of each of ten IPMNs from whom cyst fluid was available (example in FIG. 4B and C). The individual locule walls generally appeared to be monoclonal, as more than one KRAS mutation was only found in one (4.5%) of the 22 locules examined. No locule wall contained more than one GNAS mutation and two adjacent locules within a single grossly distinct IPMN were more likely to contain the same KRAS or GNAS mutation than the lining epithelium from two topographically different IPMNs (p<0.05, Fisher's Exact Test for KRAS G12D, KRAS G12R and GNAS R201H mutations; P<0.1.0 for KRAS G12V and GNAS R201H mutations). All of the ten KRAS and six GNAS mutations identified in the cyst fluid could be identified in the corresponding locule walls. These data leave little doubt that the mutations in the cyst fluid are derived from the cyst locule walls and indicate that the cyst fluid provides an excellent representation of the neoplastic cells in an IPMN.

### EXAMPLE 5

GNAS Imitations in Invasive Cancers Associated with IPMNs

Prior whole exome sequencing had not revealed any GNAS mutations in 24 typical PDA that occurred in the absence of an associated IPMN (29). We extended these data by examining 95 additional surgically resected PDAs in pancreata without evidence of IPMNs for mutations in GNAS R201H R201C, using the ligation assay described above. Again, no GNAS mutations were identified in PDAs arising in the absence of IPMNs.

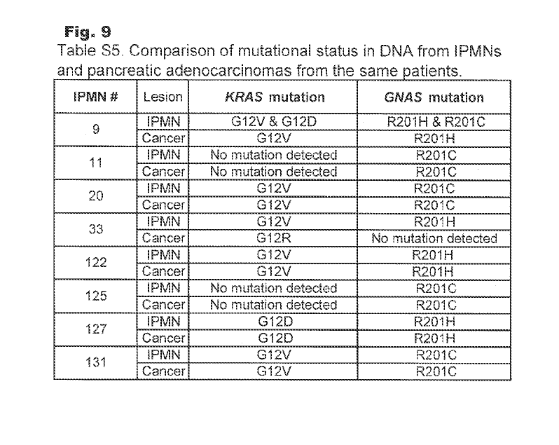

We suspected that IPMNs containing GNAS mutations had the potential to progress to an invasive carcinoma because fluids from IPMNs with high-grade dysplasia contained such mutations (Table 1). However, in light of the multiocular and multifocal nature of IPMNs described above, it was not clear whether the cells of the locule(s) that progress to an invasive carcinoma, were those that contained GNAS mutations. To address this question, we purified DNA from invasive pancreatic adenocarcinomas that developed in association with IPMNs. In each case, the neoplastic cells of the IPMN and of the invasive adenocarcinoma were carefully microdissected. In seven of the eight patients, the identical GNAS mutation found in the neoplastic cells of the IPMN was found in the concurrent invasive adenocarcinoma (FIG. 9 (Table S5)). The KRAS mutational status of the PDA was consistent with that of the associated IPMN in the same seven eases. In the eighth case, the KRAS and GNAS mutations identified in the neoplastic cells of the IPMN were not found in the associated PDA, suggesting that this invasive cancer arose from a separate precursor lesion (FIG. 9 (Table S5)). Though KRAS mutations were found commonly in both types of PDAs, there was a dramatic difference between the prevalence of GNAS mutations in PDAs associated with IPMNs (7 of 8) vs. that in PDAs unassociated with IPMNs (0 of 116; p<0.001, Fisher's Exact Test).

### EXAMPLE 6

### A Protocol for Enrichment on Beads

Cleave Oligos from the Chip

Place the chip into the corner of a Micro-Seal bag (Model 50068, DAZEY corporation cut to ˜10.5×5.5 cm.

Seal the unsealed two sides so that the bag ends up 8 cm×2.6 cm, tightly wrapping the chip.

While in the Seal-a-Meal bag, treat for five hours with 3 ml 28% ammonium hydroxide at room temperate by rotator (360 deg rotation). (Make sure the chip is fully immersed in the solution)

Transfer the solution into two 2-ml eppendorf tubes, and speed vaccum dried at temperate 50° C. (normally it will take 5-8 hours)

(For speed vaccum, turn on the cooler one hour before you use the vaccum)

Re-dissolve the oligos in a combined 400 ul RNase and DNase free water.

### Amplify the Oligos

Make 3×50 ul PCR mix for each chip, the PCR mix contains the following:

X ul H2O

1 ul (well 1), 2 ul (well 2), 5 ul (well 3)

2 ul forward primer (25 uM):
5′-TGATCCCGCGACGA*C-3′,
where * indicates phosphorothioate
2 ul reverse primer (25 uM):
5′-GACCGCGACTCCAG*C-3′,
where * indicates phosphorothioate

4 ul MgCl2 (50 mM)

5 ul 10× Platinum Tag buffer (Life Technologies)

4 ul dNTPs (10 mM each)

1 ul Platinum Tag (5 U/ul. Life Technologies) (Titanium and Phusion both did not work).

Note: Because of the alkalis condition after cleavage, the more template you add, the less PCR product you get.

The cycling conditions were: 1× 98° C. for 30 s
35 cycles of 98° C. for 30 s, 40° C. for 30 s, 60° C. for 15 s, 72° C. for 45 s
one cycle of 72° C. for 5 min

Run the gel to see a smear from 60 bp to 120 bp. 120 bp product may be dimers, which Won't interfere with capture.

The PCR products were combined, and add 2 ul Sodium Acetate (3M, pH 5.2) purified using a MinElute Purification Column (Qiagen), elute twice in 65° C. pre-warmed buffer with 17 ul each (total of 34 ul).

### End-Repair the PCR Product

End-repair using End-IT DNA End-Repair Kit (Epicentre) as follows:

34 ul DNA

5 ul 10× End-Repair Buffer

5 ul dNTP

5 ul ATP

1 ul End-Repair Enzyme Mix

Incubate at room temperature for 45 minutes,

Purified using a MinElute Purification Column (Qiagen), elute twice in 65° C. pre-warmed buffer with 17.5 ul each (total of 35 ul).

### Ligate the PCR Product

The PCR products were ligated to form concatamers using the following protocol:

35 ul End-Repaired DNA product

40 ul 2× T4 DNA ligase buffer (Enzymatics Inc.)

5 ul T4 DNA ligase (600 units/ul; Enzymatics Inc.)

The mix was incubated at room temperature for at least 4 hours, (you can leave it overnight.)

The product was purified using QiaQuick PCR Purification Column (Qiagen) (not MinElute), elute twice in 65° C. pre-warmed buffer with 25 ul each (total of 50 ul).

Quantify by absorption at 260 nm. (Normally you get around 3 ug DNA product.)

Dilute the product to 20 ng/ul using TE buffer.

Isothermal Amplification of the Probe with Bio-dUTP [RepliG-Midi Kit (not Mini Kit), Qiagen]

TABLE 1
Preparation of Buffer D1 (Volumes given are suitable
for up to 15 reactions)

| Component | Volume |
|---|---|
| Reconstituted Buffer DLB | 9 μl |
| Nuclease-free water | 32 μl |
| Total volume | 41 μl |

TABLE 2
Preparation of Buffer N1 (Volumes given are suitable for
up to 15 reactions)

| Component | Volume |
|---|---|
| Stop solution | 12 μl |
| Nuclease-free water | 68 μl |
| Total volume | 80 μl |

Place 2.5 μl template DNA into a microcentrifuge tube.

Add 2.5 μl Buffer D1 to the DNA. Mix by vortexing and centrifuge briefly

Incubate the samples at room temperature. (15-25° C.) for 3 min

Add 5 d Buffer N1 to the samples. Mix by vortexing and centrifuge briefly.

Prepare a master mix on ice according to Table 3 (see below). Mix and centrifuge briefly.

Important: Add the master mix components in the order listed in Table 3. After addition of water and REPLI-g Midi Reaction Buffer,
briefly vortex and centrifuge the mixture before addition of REPLI-g Midi DNA Polymerase. The master mix should be kept on ice and used

immediately upon addition of the REPLI-g Midi DNA Polymerase.

TABLE 3
Preparation of Master Mix

| Component | Volume/reaction |
|---|---|
| REPLI-g Midi Reaction Buffer | 14.5 μl |
| Biotin-dUTP(1 mM) (Cat. No. 11093070910, | 2.5 ul |
| Roche Applied Science) | |
| REPLI-g Midi DNA Polymerase | 0.5 μl |
| Total volume | 17.5 μl |

Add 17.5 ul of the master mix to 10 μl denatured DNA that was neutralized with N1 as described above. Transfer the mix to the PCR plate.

Incubate at 30° C. for 16 h in PCR machine.

Inactivate REPLI-g Midi DNA Polymerase by heating the sample at 65° C. for 3 min.

Transfer the mix using 2×120 ul TE to a 1.5 ml tube.

Incubate the tube in 100° C. heating block for 20 minutes.

Purify the product using two QiaQuick PCR Purification Columns (Qiagen) (not MinElute), i.e., use 2 columns for one 27.5 ul reaction.

Elute each column twice with 65° C. pre-warmed buffer with 27.5 ul, for a total of 55 ul, so there will be 110 ul of eluate from the two columns which should be pooled.

Quantify by absorption at 260 nm using nanodrop (I know it's single-strand DNA now, but I still use ds-DNA calcualtions in nanodrop)

In general, you will ˜180-210 ng/ul. If it's too off, there must be something wrong.

DNA Capture

A mix was prepared as follows:

4 ug DNA library (20 ul, 200 ng/ul)

7 ul Human cot-1 DNA (Cat.No.15279011, Invitrogen)

3 ul Herring Sperm DNA (Cat.No.15634-017, Invitrogen)

10 ul Blocking Oligos, 1 nmol/ul each.

Block Oligo 1:
AATGATACGGCGACCACCGAGATCTACACTCTTTCCCTACACGACGCTCT
Block Oligo 2:
CAAGCAGAAGACGGCATACGAGATCGGTCTCGGCATTCCTGCTGAACCGC

5 ul Capture Probe (˜200 ng/ul)

The mix is heated at 95° C. for 7 min, and 65° C. for 2 min (use only one compress pad in PCR machine)

Add 25 ul of the 65° C. prewarmed 2.8× hybridization buffer (final cone of hyb buffer will then be 1×)

2.8× hybridization buffer: (14×SSPE, 14×Denhardts, 14 mM EDTA, 0.28% SDS), using the following reagents:

20×SSPE: (0810-4L, AMRESCO)

Denhardt's Solution, 50×, 50 ml (70468, usb)

EDTA: 0.5 M, PH 8.0 (46-034-CI, Mediatech Inc.) v (In case the DNA library cone is <200 ng/ul, then still use 4 ug DNA and 7 ul Cot-1, 3 ul Herring sperm, etc. but use proportionally larger volumes of 2.8×HybBuffer

Incubate at 65 deg for 22 hours for hybridization with PCR machine lid heat on.

Washing Procedure

Wash 50 ul MyOne beads (Invitrogen) 3 times in 1.5 ml tule and resuspend in 60 μl 1× binding buffer (1 M NaCl, 10 mM Tris-HCl, pH 7.5, and 1 mM EDTA.)

Add equal volume (70 ul) of 2× binding buffer (2 M NaCl, 20 mM Tris-HO, pH 7.5, and 2 mM EDTA.) to hybrid mix, and transfer to tube with beads. Total Volume should be 200 ul.

Votex the mix thoroughly, And rotate 360 deg. for 1 hour at Room Temperature,

After binding, the beads are pulled down, and washed 15 minutes at RT in 0.5 ml Wash Buffer 1 (1×SSC/0.1% SDS)

Wash the beads for 15 minutes at 65° C. on a heating block with shaking, five times in 0.5 ml Wash Buffer 3 (0.1×SSC and 0.1% SDS)

Hybrid-selected DNA are resuspended in 50 μl 0.1 M NaOH at RT for 10 min.

The beads are pulled down, the supernatant transferred to a tube containing 70 μl Neutralizing Buffer (1 M Tris-HCl, pH 7.5)

Neutralized DNA is desalted and concentrated on a QIAquick MinElute column and eluted in 20 μl.

Note: Wash Buffer 2 (5.2 M Betaine, 0.1×SSC and 0.1% SDS) is a more stringent wash buffer.

For more stringent wash, you can substitute the first WB3 wash with WB2, then continue with four washes with WB3.

Change the post-Capture amplification Cycle number to 16 cycles if you use a more stringent wash.

Post-Capture Amplification

PCR mix containing:

20 captured DNA

51 ul dH2O

20 ul 5× Phusion buffer

5 ul DMSO

2 ul 10 mM dNTPs

0.5 ul (50 pmol) Illumina forward primer (QC1 primer for barcoding)

0.5 ul (50 pmol) Illumina reverse primer (Barcoding reverse primers for barcoding)

1 ul Hotstart Phusion enzyme (New England Biolabs)

The cycling conditions were: 1×98° C. for 30 s
14 cycles of 98° C. for 25 s, 65° C. for 30 s, 72 ° C. for 30 s
one cycle of 72° C. for 5 min

The PCR is done in two wells for each sample, 50 ul each (no oil on top).

The amplified PCR product was purified using a NucleoSpin column (Macherey Nagel, inc.). eluted twice in 65° C. pre-warmed buffer with 17.5 ul (total of 35 ul).

Use NanoDrop to quantify yield, which should be ˜20 ng/ul.

### REFERENCES

1. The disclosure of each reference cited is expressly incorporated herein. T. A. Laffan, K. M. Horton, A. P. Klein, B. Berlanstein, S. S. Siegelman, S. Kawamoto, P. T. Johnson, E. K. Fishman, R. H. Hruban, Prevalence of unsuspected pancreatic cysts on MDCT, AJR Am J Roentgenol 191, 802-807 (2008).
2. K. de Jong, C. Y. Nin, J. J. Hermans, M. G. Dijkgraaf, D. J. Goutna, C. H. J. van Eijck, E. van Heel, G. Klass, P, Fockens, M. J. Bruno, High Prevalence of Pancreatic Cysts Detected by Screening Magnetic Resonance Imaging Examinations. Clinical Gastroenterology and Hepatology 8, 806-811 (2010).
3. W. Kimura, H. Nagai, A. Kuroda, Muto, Y. Esaki, Analysis of small cystic lesions of the pancreas. Int J Pancreatol 18, 197-206 (1995).
4. K. S. Lee, A. Sekhar, N. M. Rofsky, I. Pedrosa, Prevalence of incidental pancreatic cysts in the adult population on MR imaging. Am J Gastroenterol 105, 2079-2084 (2010).
5. H. Matthaei, R. D. Schulick, R. H. Hruban, A. Maitra, Cystic precursors to invasive pancreatic cancer. Nature Reviews Gastroenterology & Hepatology 8, 141-150 (2011),
6. M. Katz, M. Mortenson, H. Wang, R. Hwang, E. Tamm, G. Staerkel, J. Lee, D. Evans, J. Fleming, Diagnosis and Management of Cystic Neoplasms of the Pancreas: An Evidence-Based Approach. Journal of the American College of Surgeons 207, 106-120 (2008).
7. M. Tanaka, Controversies in the management of pancreatic IPMN. Nature Reviews Gastroenterology & Hepatology 8, 56-60 (2011),
8. R. H. Hruban, M. B. Pitman, D. S. Klimstra, Tumors of the pancreas. Atlas of tumor pathology (American Registry of Pathology and Armed Forces Institute of Pathology, Washington, DC, ed. Fourth Series, Fascicle 6, (2007).
9. G. Klöppel, M. Kosmahl, Cystic Lesions and Neoplasms of the Pancreas. Pancreatology 1, 8 (2001).
10. M. Tanaka, S. Chari, V. Adsay, C. Fernandez-del Castillo, M. Fakoni, Shimizu, K. Yamaguchi, K. Yamao, S. Matsuno, International consensus guidelines for management of intraductal papillary mucinous neoplasms and mucinous cystic neoplasms of the pancreas. Pancreatology 6, 17-32 (2006).
11. T. A. Sohn, C. J. Yeo, J. L. Cameron, R. H. Hruban, N. Fukushima, K. A. Campbell, K. D. Lillemoe, Intraductal papillary mucinous neoplasms of the pancreas: an updated experience. Ann Surg 239, 788-797; discussion 797-789 (2004).
12. S. Crippa, C. Fernández-del Castillo, R. Salvia, D. Finkelstein, C. Bassi, I. Dominguez, A. Muzikansky, S. P. Thayer, M. Falconi, M. Mino-Kenudson, Mucin-Producing Neoplasms of the Pancreas: An Analysis of Distinguishing Clinical and Epidemiologic Characteristics. Clinical Gastroenterology and Hepatology 8, 213-219.e214 (2010).
13. G. A. Poultsides, S. Reddy, J. L. Cameron, R. H. Hruban, T. M. Pawlik, N. Ahuja, A. Jain, B. H. Edit, C. A. Iacobuzio-Donahue, R. D. Schulick, C. L. Wolfgang, Histopathologic basis for the favorable survival after resection of intraductal papillary mucinous neoplasm-associated invasive adenocarcinoma of the pancreas. Ann Surg 251, 470-476 (2010).
14. T. A. Sohn, C. J. Yeo, J. L. Cameron, L. Koniaris, S. Kaushal, R. A. Abrams, P. I. Sauter, J. Coleman, R. H. Hruban, K. D. Lillemoe, Resected adenocarcinoma of the pancreas 616 patients results, outcomes, and prognostic indicators. Journal of Gastrointestinal Surgery 4, 13 (2000).
15. R. Salvia, C. Fernandez-del Castillo, C. Bassi, S. P. Thayer, M. Falconi, W. Mantovani, P. Pederzoli, A. L. Warshaw, Main-duct intraductal papillary mucinous neoplasms of the pancreas: clinical predictors of malignancy and long-term survival following resection. Ann Surg 239, 678-685; discussion 685-677 (2004),
16. P. U. Freda, W. K. Chung, N. Matsuoka, J. E. Walsh, M. N. Kanibir, G. Kleinman, Y, Wang, J. N. Bruce, K. D. Post, Analysis of GNAS mutations in 60 growth hormone secreting pituitary tumors: correlation with clinical and pathological characteristics and surgical outcome based on highly sensitive GH and IGF-I criteria for remission. Pituitary 10, 275-282 (2007).
17. N. Kalfa, Activating Mutations of the Stimulatory G Protein in Juvenile Ovarian Granulosa Cell Tumors: A New Prognostic Factor? Journal of Clinical Endocrinology & Metabolism 91, 1842-1847 (2006).
18. M. C. Fragoso, A. C. Latronico, F. M. Carvalho, M. C. Zerbini, J. A. Marcondes, L. M. Araujo, V. S. Lando, E. T. Frazzatto, B. B. Mendonca, S. M. Villares, Activating mutation of the stimulatory protein (gsp) as a putative cause of ovarian and testicular human stromal Leydig cell tumors. J Endocrinol Metab 83, 2074-2078 (1998),
19. H. Yamasaki, N. Mizusawa, S. Nagahiro, S. Yamada, I. Sano, M. Itakura, K. Yoshimoto, GH-secreting pituitary adenomas infrequently contain inactivating mutations of PRKAR1A and LOH of 17q23-24. Clin EndocrinoI (Oxf) 58, 464-470 (2003).
20, L. D. Wood, D. W. Parsons, S. Jones, J. Lin, T. Sjoblom, R. J. Leary, D. Shen, S. M. Boca, T. Barber, J. Ptak, N. Silliman, S. Szabo, Z. Dezso, V. Ustyanksky, T. Nikolskaya, Y. Nikolsky, R. Karchin, P. A. Wilson, J. S. Kaminker, Z. Zhang, R. Croshaw, J. Willis, D. Dawson, M. Shipitsin, J. K. V. Willson, S. Sukumar, K. Polyak, B. H. Park, C. L. Pethiyagoda, P, V. K. Pant, D. 0. Ballinger, A. B. Sparks, J. Hartigan, D. R. Smith, E. Sub, N. Papadopoulos, P. Buckhaults, S. D. Markowitz, G. Parmigiani, K. W. Kinzler, V. E. Velculescu, B. Vogelstein, The Genomic Landscapes of Human Breast and Colorectal Cancers. Science 318, 1108-1113 (2007).
21. S. Idziaszczyk, C. H. Wilson, C. G. Smith, D. J. Adams, J. P. Cheadle, Analysis of the frequency of GNAS codon 201 mutations in advanced colorectal cancer. Cancer Genetics and Cytogenetics 202, 67-69 (2010).
22. J.-S. Shin, A. Spillane, E. Wills, W. A. Cooper, PEComa of the retroperitoneum. Pathology 40, 93-95 (2008).
23. I. J. Dahabreh, T. Terasawa, P. J. Castaldi, T. A. Trikalinos, Systematic review: Anti-epidermal growth factor receptor treatment effect modification by KRAS mutations in advanced colorectal cancer. Ann Intern Med 154, 37-49 (2011).
24. C. Almoguera, D. Shibata, K. Forrester, J. Martin, N. Arnheim, M. Perucho, Most human carcinomas of the exocrine pancreas contain mutant c-K-ras genes. Cell 53, 549-554 (1988).
25. S. Fritz, C. Fernandez-del Castillo, M. Mino-Kenudson, S. Crippa, V. Deshpande, G. Y. Lauwers, A. F. Warshaw, S. P. Thayer, A. J. Iafrate, Global Genomic Analysis of Intraductal Papillary Mucinous Neoplasms of the Pancreas Reveals Significant Molecular Differences Compared to Ductal Adenocarcinoma. Annals of Surgery 249, 440-447 (2009).
26. D. Soldini, M. Gugger, E. Burckhardt, A. Kappeler, J. A. Laissue, L. Mazzucchelli, Progressive genomic alterations in intraductal papillary mucinous tumours of the pancreas and morphologically similar lesions of the pancreatic ducts. The Journal of Pathology 199, 453-461 (2003).
27. F. Schönleben, W. Qiu, K. C. Bruckman, N. T. Ciau, X. Li, M. EL Lauerman, H. Frucht, J. A. Chabot, J. D. Allendorf, H. E. Remotti, BRAF and KRAS gene mutations in intraductal papillary mucinous neoplasm/carcinoma (IPMN/IPMC) of the pancreas. Cancer Letters 249, 242-248 (2007).
28. K. Wada, Does “clonal progression” relate to the development of intraductal papillary mucinous tumors of the pancreas? Journal of Gastrointestinal Surgery 8, 289-296 (2004).
29. S. Jones, X. Zhang, D. W. Parsons, J. C. H. Lin, R. J. Leary, P. Angenendt, P. Mankoo, H. Carter, H. Kamiyama, A. Jimeno, S. M. Hong, B. Fu, M. T. Lin, E. S. Calhoun, M. Kamiyama, K. Walter, I. Nikolskaya, Y. Nikolsky, J. Hartigan, D. R. Smith, M. Hidalgo, S. D. Leach, A. P. Klein, E. M. Jaffee, M. Goggins, A. Maitra, C. Iacobuzio-Donahue, J. R. Eshleman, S. E. Kern, R. H. Hruban, R. Karchin, N. Papadopoulos, G. Parmigiani, B. Vogelstein, V. E. Velculescu, K. W. Kinzler, Core Signaling Pathways in Human Pancreatic Cancers Revealed by Global Genomic Analyses. Science 321, 1801-1806 (2008).
30. G. Parmigiani, S. Boca, J. Lin, K. W. Kinzler, V. Velculescu, B. Vogelstein, Design and analysis issues in genome-wide somatic mutation studies of cancer, Genomics 93, 17-21 (2009).
31. F. Schonleben, J. D. Allendorf, W. Qui, X. Li, D. J. Ho, N. T. Ciau, R. L. Fine, J. A. Chabot, H. E. Remotti, G. H. Su, Mutational analyses of multiple oncogenic pathways in intraductal papillary mucinous neoplasms of the pancreas. Pancreas 36, 168-172 (2008).
32. M. Kitago, M. Ueda, K. Aiura, K. Suzuki, S. Hoshimoto, S. Takahashi, M. Mukai, M. Kitajima, Comparison of K-ras point mutation distributions in intraductal papillary-mucinous tumors and ductal adenocarcinoma of the pancreas, International Journal of Cancer 110, 177-182 (2004).
33. T. Izawa, T. Obara, S. Tanno, Y. Mizukami, N. Yanagawa, Y. Kohgo, Clonality and Field Cancerization in Intraductal Papillary-Mucinous Tumors of the Pancreas. Cancer 92, 11 (2001),
34. F. Diehl, M. Li, He, K. W. Kinzler, B. Vogelstein, D. Dressman, BEAMing: single-molecule PCR on microparticles in water-in-oil emulsions. Nature Methods 3, 551-559 (2006).
35. H. Fujii, M. Inagaki, S. Kasai, N. Miyokawa, Y. Tokusashi, E. Gabrielson, R. H. Hruban, Genetic progression and heterogeneity in intraductal papillary-mucinous neoplasms of the pancreas. American Journal of Pathology 151, 8 (1997).
36. B. Taouli, V. r. Vilgrain, M.-P. Vullierme, B. t. Terris, A. Denys, A. Sauvanet, P. Hammel, Y. Menu, Intraductal Papillary Mucinous Tumors of the Pancreas: Helical CT with Histopathologic Correlation. Radiology 217, 8 (2000).
37. A. Diaz, M. Danon, J. Crawford McCune-Albright syndrome and disorders due to activating mutations of GNAS1. J Pediatr Endocrinol Metab 20, 853-880 (2007).
38. A. Lania, A. Spada, G-protein and signalling in pituitary tumours. Horm Res 71 Suppl 2, 95-100 (2009).
39. A. G. Lania, G. Mantovani, A. Spada, Mechanisms of disease: Mutations of G proteins and G-protein-coupled receptors in endocrine diseases. Nat Clin Pract Endocrinol Metab 2, 681-693 (2006).
40. D. Shibata, J. Schaeffer, Z. H. Li, G. Capella, M. Perucho, Genetic heterogeneity of the c-K-ras locus in colorectal adenomas but not in adenocarcinomas. J Natl Cancer Inst 85, 1058-1063 (1993).
41. S. Jones, W. D. Chen, G. Parmigiani, F. Diehl, N. Beerenwinkel, T. Antal, A. Traulsen, M. A. Nowak, C. Siegel, V. E. Velculescu, K. W. Kinzler, B. Vogelstein, J. Willis, S. D. Markowitz, Comparative lesion sequencing provides insights into tumor evolution. Proc Natl Acad Sci U S A 105, 4283-4288 (2008).
42. C. Correa-Gallego, C. R. Ferrone, S. P. Thayer, J. A. Wargo, A. L. Warshaw, C. Fernandez-del Castillo, Incidental Pancreatic Cysts: Do We Really Know What We Are Watching? Pancreatology 10, 144-150 (2010).
43. J. F. Tseng, A. L. Warshaw, D. V. Sahani, G. Y. Lauwers, D. W. Rattner, C. F.-d. Castillo, Serous Cystadenoma of the Pancreas. Transactions of the . . . Meeting of the American Surgical Association 123, 111-118 (2005).
44. S.-M, Hong, D. Kelly, M. Griffith, N. Omura, A. Li, C.-P. Li, R. H. Hruban, M. Goggins, Multiple genes are hypermethylated in intraductal papillary mucinous neoplasms of the pancreas. Mod Pathol 21, 9 (2008).
45. P. J. Allen, L.-X. Qin, L. Tang, D. Klimstra, M. F. Brennan, A. Lokshin, Pancreatic Cyst Fluid Protein Expression Profiling for Discriminating Between Serous Cystadenoma and Intraductal Papillary Mucinous Neoplasm. Annals of Surgery 250, 754-760 (2009).
46. E. Ke, B. B. Patel, T. Liu, X.-M. Li, O. Haluszka, J. P. Hoffman, H. Ehya, N. A. Young, J. C. Watson, D. S. Weinberg, M. I. Nguyen, S. J. Cohen, N. J. Meropol, S. Litwin, J. L. Tokar, A. T. Yeung, Proteomic Analyses of Pancreatic Cyst Fluids. Pancreas 38, 10 (2009).
47. A. Khalid, M. Zahid, S. D. Finkelstein, J. K. LeBlanc, N. Kaushik, N. Ahmad, W. R. Brugge, S. A. Edmundowicz, R. H. Hawes, K. M. McGrath, Pancreatic cyst fluid DNA analysis in evaluating pancreatic cysts: a report of the PANDA study. Gastrointest Endosc 69. 1095-1102 (2009).
48. M. S. Sawhney, S. Devarajan, P. O'Farrel, M. S. Cury, R. Kundu, C. M. Vollmer, A. Brown, R. Chuttani, D. K. Pleskow, Comparison of carcinoembryonic antigen and molecular analysis in pancreatic cyst fluid. Gastrointestinal Endoscopy 69, 1106-1110 (2009).
49. K. E. Schoedel, S. D. Finkelstein, N. P. Ohori, K-Ras and microsatellite marker analysis of fine-needle aspirates from intraductal papillary mucinous neoplasms of the pancreas, Diagnostic Cytopathology 34, 605-608 (2006).
50. D. Bartsch, D. Bastian, P. Barth, A. Schudy, C. Nies, O. Kisker, H. J. Wagner, M. Rothmund, K-ras oncogene mutations indicate malignancy in cystic tumors of the pancreas. Ann Surg 228, 79-86 (1998).
51. M. Al-Haddad, M. B. Wallace, T. A. Woodward, S. A. Gross, C. M. Hodgens, R. D. Toton, M. Raimondo, The safety of fine-needle aspiration guided by endoscopic ultrasound: a prospective study. Endoscopy 40, 204-208 (2008).
52. D. V. Sahani, R. Kadavigere, A. Saokar, C. Fernandez-del Castillo, W. R. Brugge, P. F. Hahn, Cystic pancreatic lesions: a simple imaging-based classification system for guiding, management. Radiographics 25, 1471-1484 (2005).
53. F. T. Bosman, F. Carneiro, R. H. Hruban, N. D. Thiese, WHO Classification of Tumours of the Digestive system. (IARC Press, Lyon, ed. 4, 2010), vol. 3.
54. T. Furukawa, G. Kloppel, N. Volkan Adsay, J. Albores-Saavedra, N. Fukushima, A. Horii, R, H. Hruban, Y. Kato, D. S. Klimstra, D. S. Longnecker, J. Lunges, G. J. Offerhaus, M. Shimizu, M. Sunamura, A. Suriawinata, K. Takaori, S. Yonezawa, Classification of types of intraductal papillary-mucinous neoplasm of the pancreas: a consensus study. Virchows Arch 447, 794-799 (2005).
55. C. Rago, D. L. Huso, F. Diehl, B. Karim, G. Liu, N. Papadopoulos, Y. Samuels, V. E. Veleulescu, B. Vogelstein, K. W. Kinzler, L. A. Diaz, Jr., Serial Assessment of Human Tumor Burdens in Mice by the Analysis of Circulating DNA. Cancer Res 67, 9364-9370 (2007).
56. F. Diehl, K. Schmidt, M. A. Choti, K. Romans, S. Goodman, M. Li, K. Thornton, N. Agrawal, L. Sokoll, S. A. Szabo, K. W. Kinzler, B. Vogelstein, L. A. Diaz Jr, Circulating mutant DNA to assess tumor dynamics. Nature Medicine 14, 985-990 (2007).
57, D. S. Herman, G. K. Hovingh, O. Iartchouk, H. L. Rehm, R. Kucherlapati, J. G. Seidman, C. E. Seidman, Filter-based hybridization capture of subgenomes enables resequencing and copy-number detection. Nature Methods 6, 507-510 (2009).
58, C. Fouquet, M. Antoine, P. Tisserand, R. Favis, M. Wislez, F. Commo, N. Rabbe, M. F. Carette, B. Milleron, F. Barany, J. Cadranel, G. Zalcman, T. Soussi, Rapid and sensitive p53 alteration analysis in biopsies from lung cancer patients using a functional assay and a universal oligonucleotide array: a prospective study. Clin Cancer Res 10, 3479-3489 (2004).
59. S. M. Doug, G. Traverso, C. Johnson, L. Geng, R. Favis, K. Boynton, K. Hibi, S. N. Goodman, M. D'Allessio, P. Paty, S. R. Hamilton, D, Sidransky, F. Barany, B. Levin, A. Shuber, K. W. Kinzler, B. Vogelstein, J. Jen, Detecting Colorectal Cancer in Stool With the Use of Multiple Genetic Targets. J Natl Cancer Inst 93, 858-865. (2001).
60. J. Luo, D. E. Bergstrom, F. Barany, Improving the fidelity of Thermus thermophilus DNA ligase. Nucleic Acids Res 24, 3071-3078 (1996).
61. C. Shi, S. H. Eshleman, D. Jones, N. Fukushima, L. Hua, A. R. Parker, C. J. Yeo, R. H. Hruban, M. G. Goggins, J. R. Eshleman, LigAmp for sensitive detection of single-nucleotide differences. Nat Methods 1, 141-147 (2004).
62. F. Diehl, K. Schmidt, M. A. Choti, K. Romans, S. Goodman, M. Li, K. Thornton, N. Agrawal, L. Sokoll, S. A. Szabo, K. W. Kinzler, B. Vogelstein, L. A. Diaz, Jr., Circulating mutant DNA to assess tumor dynamics. Nat Med. 14, 985-990 (2008).
63. T. S. L. StataCorp. 2009. Stata Statistical Software: Release 11. College Station.

1. A method for detecting mutations at a selected location in a nucleotide sequence, comprising the steps of:
contacting to form a reaction mixture:
(a) a test sample comprising 200 or fewer molecules of analyte nucleic acid;
(b) a probe complementary to a wild-type sequence at the selected location and adjacent to and proximal to the selected location;
(c) a probe complementary to a mutant sequence at the selected location and adjacent to and proximal to the selected location;
(d) an anchoring oligonucleotide which is complementary to e analyte nucleic acid adjacent to and distal to the selected location; and
(e) thermotolerant DNA ligase;
wherein the probes complementary to the wild-type and mutant sequences are labeled with distinct fluorescent moieties, or wherein the probes complementary to the wild-type and mutant sequences are of distinct lengths, or wherein the probes complementary to the wild-type and mutant sequences have distinct fluorescent moieties and distinct lengths;
thermocycling the reaction mixture such that anchoring oligonucleotides are ligated to an appropriate probe reflecting hybridization of the appropriate probe to the analyte nucleic acid, thereby forming ligation products;
separating the ligation products on a gel, or detecting the distinct fluorescent moieties, or separating the ligation products on a gel and detecting the distinct fluorescent moieties on the separated ligation products on the gel.

2. The method of claim 1 the test sample is an amplification product.

3. The method of claim 1 further comprising the step of: asymmetrically amplifying an analyte nucleic acid with a first and second primer, wherein the first primer is in excess of a second primer, to form the test sample.

4. The method of claim 1 wherein the probe complementary to the mutant sequence has a Tm of 32 to 36 deg C., the probe complementary to the wild-type sequence has a Tm of 32 to 38 deg C., and the anchoring oligonucleotide has a Tm of 36 to 44 deg C. as assessed by oligocale algorithm.

5. The method of claim 1 wherein the probe complementary to the mutant sequence comprises one or more locked nucleic acid nucleotides.

6. The method of claim 1 wherein the probe complementary to the mutant sequence comprises three locked nucleic acid nucleotides.

7. The method of claim 1 wherein the probe complementary to the mutant sequence comprises three locked nucleic acid nucleotides at positions -2,-3, and -7, wherein position 0 is the selected location.

8. The method of claim 1 wherein the probes complementary to the wild-type and mutant sequences are labeled with distinct fluorescent moieties.

9. The method of claim 1 wherein the probes complementary to the wild-type and mutant sequences are of distinct lengths.

10. The method of claim 1 wherein the probes complementary to the wild-type and mutant sequences have distinct fluorescent moieties and distinct lengths.

11. The method of claim 8 wherein the mutation is detected if the fluorescent moiety with which the probe complementary to the mutant sequence is labeled is detected.

12. The method of claim 10 Wherein the mutation is detected if the fluorescent moiety with which the probe complementary to the mutant sequence is labeled is detected.

13. A method for detecting mutations at a selected location in a nucleotide sequence, comprising the steps of:
asymmetrically amplifying an analyte nucleic acid with a first and second prix wherein the first primer is in excess of a second primer, to form a test sample;
contacting to form a reaction mixture:
(a) 200 or fewer molecules of analyte nucleic acid of the test sample;
(b) a probe complementary to a wild-type sequence at the selected location and adjacent to and proximal to the selected location;
(c) a probe complementary to a mutant sequence at the selected location and adjacent to and proximal to the selected location;
(d) an anchoring oligonucleotide Which is complementary to the analyte nucleic acid adjacent to and distal to the selected location; and
(e) thermotolerant DNA ligase;
wherein the probe complementary to the mutant sequence has a Tm of 32 to 36 deg C., the probe complementary to the wild-type sequence has a Tm of 32 to 38 deg C., and the anchoring oligonucleotide has a Tm of 36 to 44 deg C. as assessed by oligocalc algorithm, wherein the probe complementary to the mutant sequence comprises one or more locked nucleic acid nucleotides, wherein the wild-type and mutant probes are labeled with distinct fluorescent moieties, or wherein the wild-type and mutant probes are of distinct lengths, or wherein the wild-type and mutant probes have distinct fluorescent moieties and distinct lengths;
thrmocycling the reaction mixture such that anchoring oligonucleotides are ligated to an appropriate probe reflecting hybridization of the appropriate probe to the analyte nucleic acid, thereby forming ligation products;
separating the ligation products on a gel, or detecting the distinct fluorescent moieties, or separating the ligation products on a gel and detecting the distinct fluorescent moieties on the separated ligation products on the gel.

14. The method of claim 13 wherein the probes complementary the wild-type and mutant sequences are labeled with distinct fluorescent moieties.

15. The method of claim 13 wherein the probes complementary to the wild-type and mutant sequences are of distinct lengths.

16. The method of claim 13 wherein the probes complementary to the wild-type and mutant sequences have distinct fluorescent moieties and distinct lengths.

17. The method of claim 14 wherein the mutation is detected if the fluorescent moiety with which the probe complementary to the mutant sequence is labeled is detected.

18. The method of claim 16 wherein the mutation is detected if the fluorescent moiety with which the probe complementary to the mutant sequence is labeled is detected.


SEQUENCE LISTING (3 sequences)

SEQ ID NO: 1 - DNA, 60 bp, Artificial Sequence
  primers and probes
tcccgcgacgacnnnnnnnnnnnnnnnnnnnnnnnnnnnnnnnnnnnngctggagtcgcg

SEQ ID NO: 2 - DNA, 15 bp, Artificial Sequence
  primers and probes
tgatcccgcgacgac

SEQ ID NO: 3 - DNA, 15 bp, Artificial Sequence
  primers and probes
gaccgcgactccagc